(19) United States
(12) Patent Application Publication
(10) Pub. No.: US 2003/0052278 A1
(43) Pub. Date: Mar. 20, 2003

(54) Apparatus and method for sterilization of heat sensitive liquids

(76) Inventor: Raul Duarte, Belleville, IL (US)

Correspondence Address: Rebecca J. Brandau, 24th Floor, 720 Olive Street, St. Louis, MO, 63101, US

(21) Appl. No.: 09/954,840

(22) Filed: Sep. 18, 2001

(51) Int. Cl. G01N 21/01

(52) U.S. Cl. 250/438

(57) ABSTRACT

An apparatus and method for sterilizing a heat sensitive liquid by irradiation of the liquid with ultraviolet (UV) radiation includes an outer quartz tube arranged coaxially with an inner heat exchange tube to form an annular flow channel between the tubes. The coaxial tubes are contained in a cylindrical housing adapted to secure a plurality of UV lights in a ring around the coaxial tubes. As the liquid to be sterilized flows continuously through the annular flow channel, the liquid is irradiated as the UV radiation produced by the UV lights passes through the quartz tube, while the liquid is simultaneously being cooled by the heat exchange tube to prevent heat damage to the liquid.

### BACKGROUND OF INVENTION

The present invention relates generally to the sterilization of heat sensitive liquids and more particularly to an apparatus and method for sterilizing heat sensitive liquids utilizing ultraviolet (UV) radiation.

Liquids used in the pharmaceutical manufacturing of products such as tissue culture liquid media and serum are susceptible to biological contamination including viruses and bacteria. These microorganisms degrade the quality of the liquid, thus compromising the sterility of the product being manufactured. However, some of these liquids degrade or are otherwise compromised when exposed to increased temperatures, rendering conventional sterilization methods inadequate. The typical bacteria, virus or microorganism found in these liquids require an UV dose of 1 to 26 mw/sec/cm² to achieve a one log reduction in biological contaminants. The term sterilization includes any reduction in bacteria, virus or microorganism that is satisfactory for an intended application.

Known methods for sterilizing heat sensitive biological liquids include ultrafiltration methods and the use of gamma radiation. These methods are costly, time consuming and allow only batch type production. The use of gamma radiation is further complicated by the added step of freezing the biological liquid prior to radiation exposure to prevent heat damage to the liquid.

It is therefore a primary object of the present invention to provide a sterilization apparatus and method that allow sterilization of heat sensitive liquids without increasing the temperature of the liquid to the point of damaging the liquid.

It is another object of the present invention to provide an apparatus and method for sterilization of heat sensitive liquids that provide a continuous flow of sterilized liquid.

It is yet another object of the present invention to provide an apparatus and method for sterilization of heat sensitive liquids that are economical to construct and operate, and are simple to operate, clean and maintain.

### SUMMARY OF INVENTION

To accomplish the foregoing and other objects of this invention there are provided an apparatus and a method for sterilizing heat sensitive liquids. In a preferred embodiment, the apparatus is constructed of a substantially cylindrical aluminum housing, the interior of the housing being polished to a mirror finish.

The apparatus further includes inner and outer flow tubes that are arranged coaxially to form an annular flow channel in the space between the tubes. The outer tube is made from a material that is penetrable by UV radiation, preferably fused silica quartz. The inner tube is heat exchanger through which a cooling media flows, such as a stainless steel heat exchanger with a mirror finished exterior, through which water flows, for example. A liquid inlet port, a liquid outlet port; i.e. fittings with flow control valves, are in fluid communication with the annular flow channel.

A housing encases a UV radiation source that is used to sterilize the liquid, such as a series of elongated UV lights arranged substantially in a ring, to form a cylinder around the outer flow channel. The lights are each connected by conventional wiring to a control panel so that each light can be monitored individually. To provide maximum efficiency and light life, the lights are cooled by blowing ambient air through the housing.

In operation, the liquid to be sterilized is introduced into the apparatus through the fluid inlet port into the annular flow channel created between the inner and outer flow tubes. As the liquid flows continuously, the UV lights irradiate the liquid. The UV radiation produced by the lights penetrates the quartz tubing and the liquid. The mirror finish of the heat exhanger inner tube reflects the radiation back through the liquid, increasing the total radiation the liquid experiences as it flows through the apparatus. Likewise, radiation that reaches the housing is reflected back toward the liquid to be sterilized. The sterilized liquid is then removed from the apparatus through the fluid outlet port.

Accordingly, in keeping with the above objects and advantages, the present invention is, briefly, an apparatus for sterilizing a fluid having housing, a fluid channel within the housing adapted to control the temperature of the fluid; and an ultraviolet source disposed within the housing sufficiently close to the fluid channel to permit irradiation of the fluid as the fluid flows through the fluid channel.

The invention is further, briefly, apparatus for sterilizing a liquid having a housing. Inner and outer flow tubes are arranged coaxially within the housing to form an annular flow channel between the inner and outer flow tubes, the outer flow tube being adapted to allow penetration of UV radiation, and the inner tube being a heat exchanger. At least one liquid inlet port and at least one liquid outlet port are included in the apparatus, the liquid inlet port and the liquid outlet port being in fluid communication with the annular flow channel. There is also at least one UV radiation source to sterilize the liquid, the UV radiation source being located within the housing outwardly of the inner and outer flow tubes, so that the liquid to be sterilized is introduced through the inlet port into the annular flow channel, the liquid being irradiated by the UV radiation source as the heat exchanger controls the temperature of the liquid until the liquid is removed from the annular flow channel through the outlet port.

The invention is still further, briefly, an apparatus for sterilizing a heat sensitive liquid. The apparatus having a housing including a cooling mechanism, and inner and outer flow tubes arranged coaxially to form an annular flow channel between the inner and outer flow tubes. The outer flow tube is adapted to allow penetration of UV radiation, and the inner tube is a mirror finish stainless steel heat exchanger. At least one fluid inlet port and at least one fluid outlet port are in fluid communication with the annular flow channel for passage therethrough of the fluid to be sterilized; and there are a plurality of UV lights to produce UV radiation to sterilize the liquid. The UV lights are located within the housing and outside the inner and outer flow tubes; so that the liquid to be sterilized is introduced through the inlet port into the annular flow channel, the liquid being irradiated by the UV lights as the UV lights are cooled by the cooling mechanism, the heat exchanger controlling the temperature of the liquid until the liquid is removed from the annular flow channel.

The present invention is yet further, briefly, an apparatus for sterilizing a heat sensitive liquid having a substantially cylindrical housing including a cooling mechanism having at least one cooling medium inlet and at least one cooling medium outlet. Inner and outer flow tubes are arranged coaxially to form an annular flow channel between the inner and outer flow tubes, the outer flow tube being fused silica quartz, the inner tube being a mirror finish stainless steel heat exchanger, to thereby permit the UV radiation to penetrate the quartz tube to irradiate the liquid in the annular flow channel, the UV radiation being reflected by the heat exchanger back through the liquid in the annular flow channel, the heat exchanger including at least one heat exchange fluid inlet port and at least one heat exchange fluid outlet port so that a heat exchange fluid can flow through the heat exchanger to control the temperature of the liquid being sterilized. A plurality of elongated UV lights are located within the housing and arranged in a ring around the outer flow tube; and a housing support framework is adapted to position the housing so that the liquid inlet port is at a higher level than the liquid outlet port; to thereby permit the liquid to be sterilized to be introduced through the fluid inlet port into the annular flow channel, the liquid being irradiated by the UV lights, and the UV lights being cooled by the cooling medium as the heat exchanger controls the temperature of the liquid until the liquid is removed from the annular flow channel through the fluid outlet port.

The method of the present invention includes, briefly, determining sterilization parameters including an UV radiation level, a thickness for a liquid flow channel and a flow rate through the liquid flow channel necessary to sterilize the liquid; introducing the liquid into the flow channel; flowing the liquid through the flow channel at the determined flow rate; irradiating the liquid in the flow channel with an UV radiation source to sterilize the liquid while simultaneously; cooling the liquid in the flow channel to prevent degradation of the liquid; and removing the sterilized liquid from the flow channel.

The new method is also, briefly, method for sterilizing a heat sensitive liquid including: determining sterilization parameters including an UV radiation level, a thickness for a liquid flow channel and a flow rate through a liquid flow channel necessary to sterilize the liquid; introducing the liquid into the flow channel, the flow channel being formed between an outer flow tube and an inner flow tube coaxially arranged relative to one another, the outer flow tube being transparent to UV radiation and the inner flow tube being a heat exchanger; flowing the liquid through the flow channel at the determined flow rate; irradiating the liquid in the flow channel with an UV radiation source to sterilize the liquid, the UV radiation penetrating the outer flow tube, while simultaneously; cooling the liquid in the flow channel as the liquid flows over the heat exchanger to prevent heat degradation of the liquid; and removing the sterilized liquid from the flow channel.

These and other goals and advantages of the present invention will be in part apparent and in part pointed out hereinbelow.

### BRIEF DESCRIPTION OF DRAWINGS

FIG. 1 is a side perspective view of a preferred embodiment of the apparatus of the present invention.

FIG. 2 is a top perspective view of the apparatus illustrated in FIG. 1, enlarged and turned 90°.

FIG. 3 is a top perspective view of the apparatus illustrated in FIG. 2 with the housing opened to show an arrangement of the UV lights.

FIG. 4 is a longitudinal sectional schematic view of the apparatus of the present invention.

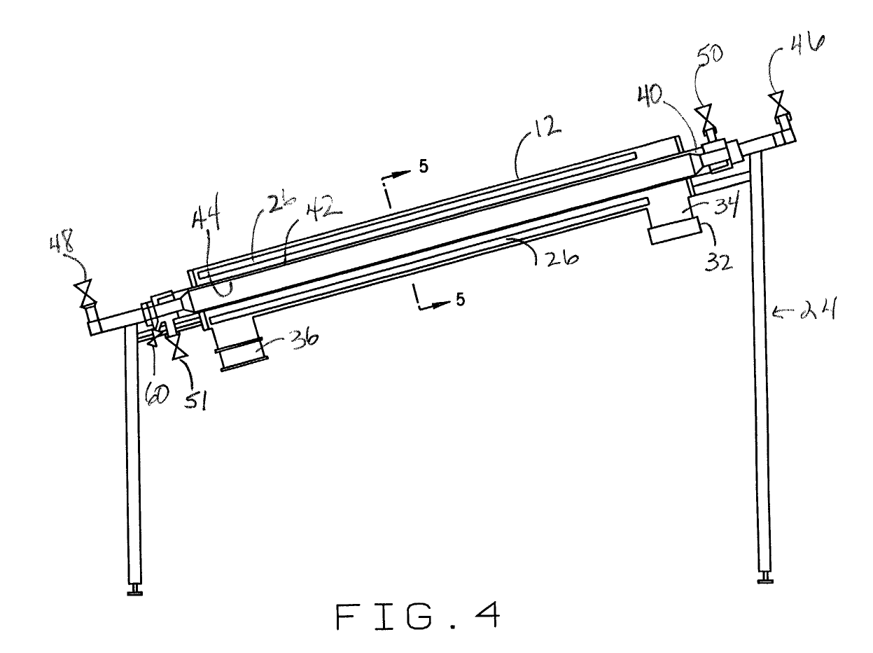

FIG. 5 is a transverse sectional view, enlarged, taken along line 5-5 of FIG. 4.

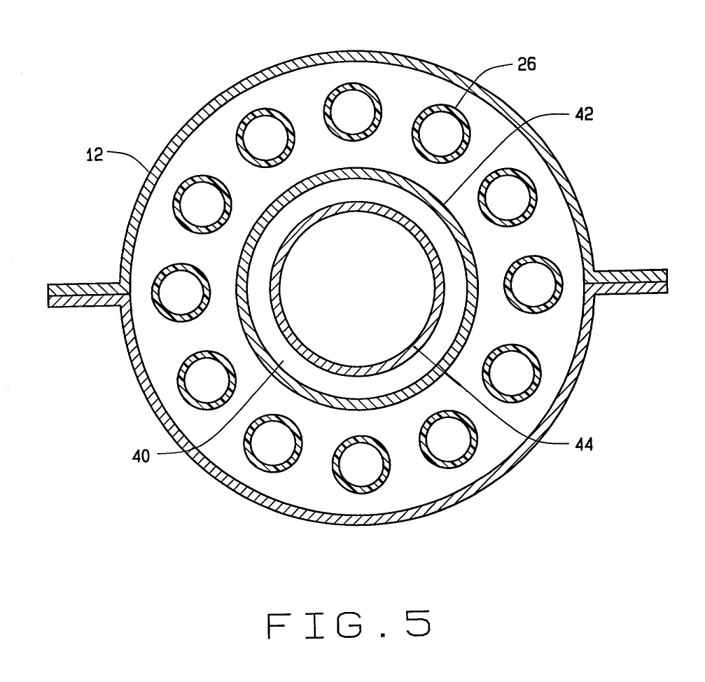

Throughout the drawings like parts are indicated by like element numbers.

### DETAILED DESCRIPTION

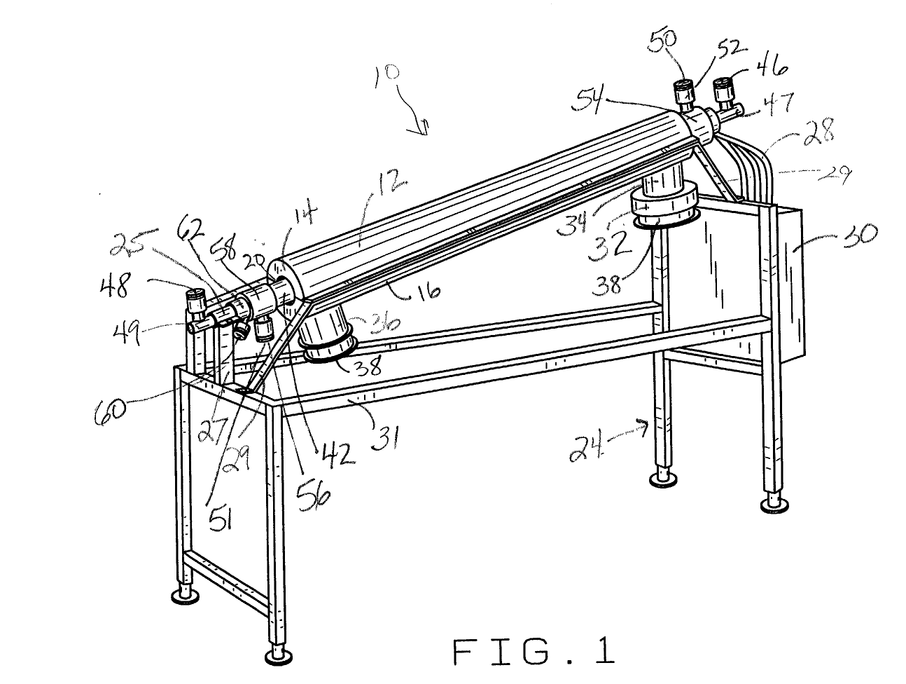

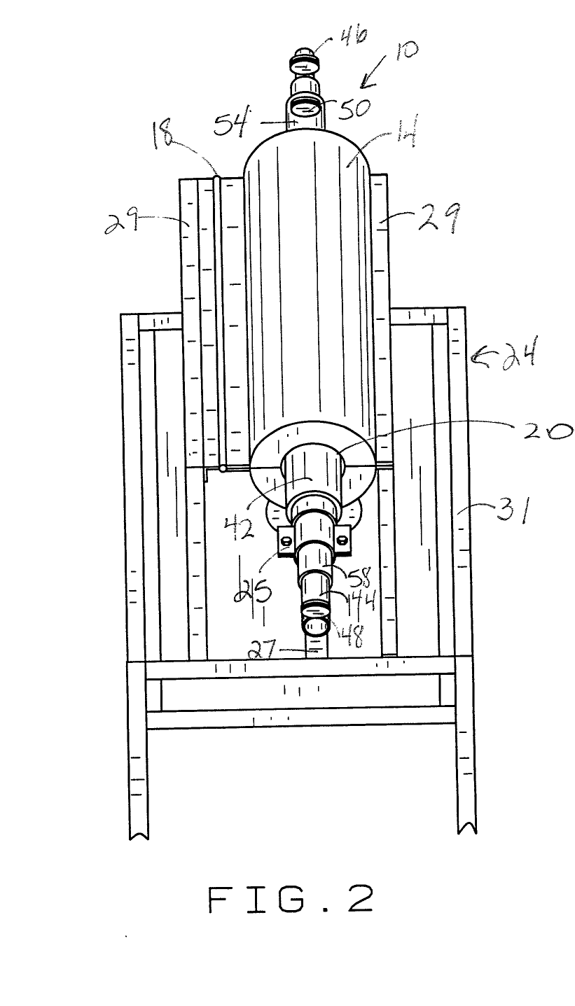

There is shown in FIGS. 1-3 a preferred embodiment of the apparatus of the present invention, generally designated 10. The apparatus 10 includes a substantially cylindrical housing 12, which is preferably, although not necessarily, formed of aluminum, and which includes a movable section 14 and a stationary section 16. Sections 14 and 16 are connected longitudinally to one another by a hinge 18. Housing 12 has opposed ends which define apertures 20, 22, and is supported on an angle by a conventional framework, generally designated 24, to which the housing is attached. Preferably at least two brackets 29 secure the housing to the base portion 31 of framework 24, although other suitable structural support connections can be conceived.

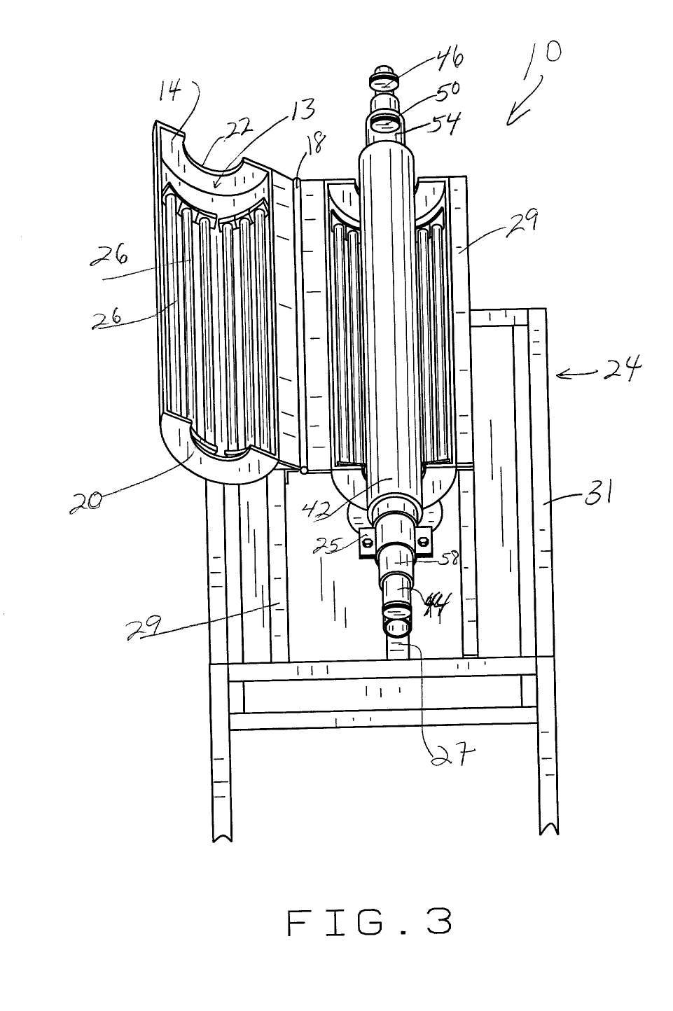

FIG. 3 illustrates that the preferably cylindrical housing 12 encases a plurality of elongated UV lights 26, which emit primarily 254 nm wavelength, and are arranged substantially in a ring, so as to form a cylinder of lights mounted around the interior 13 of the housing 12 The UV lights 26 are accessible by raising movable section 14 of the housing. A series of cables 28 power UV lights 26 and cables 28 further connect UV lights 26 to a control panel 30 that allows control and monitoring of individual lights. In the event that an individual light bums out or malfunctions, control panel 30 alerts the operator so that the light may be replaced or repaired.

Lights 26 are preferably cooled by ambient air that is blown through housing 12 by a fan 32 located within or connected to a vent inlet 34 disposed, in the embodiment illustrated, at one end of housing 12. In this embodiment the air exits the housing primarily at a vent outlet 36, other air venting arrangements can be conceived which will suffice. Lights 26 are cooled to improve the efficiency of the lights and maximize the lifetime of the lights. Ideally, the UV lights should be kept at or near a comfortable room temperature. Vents 34,36 include UV shields 38, which are desirably formed of Plexiglas, to protect the operator from UV radiation during operation of apparatus 10.

An annular flow channel 40 is created by the space formed between an outer flow tube 42 arranged coaxially around an inner flow tube 44. Outer flow tube 42 and inner flow tube 44 are located substantially centrally within housing 12, coaxially therewith, and exit the housing at apertures 20,22. Outer flow tube 42 and inner flow tube 44 are secured to base 31 by at least one collar 25 attached to a corresponding support 27 mounted to framework 24.

In a preferred embodiment the diameter of annular space of the flow channel (tube) 40 is two mm thick. In determining the optimum wall thickness of flow channel 40, factors which must be taken into account include the maximum thickness through which the UV radiation can travel, determined by the opacity of the liquid, as well as the cooling capacity of the heat exchanger. Outer flow tube 42 is preferably made of fused silica quartz, and is therefore penetrable by UV radiation. Inner flow tube 44 is a heat exchanger, preferably formed of a stainless steel heat exchange tube with a mirror finish. Inner flow tube/heat exchanger 44 has an inlet port 46 and an outlet port 48 mounted at opposed ends of the exchanger, to facilitate the flow of a cooling medium, preferably water, through inner tube 44.

A liquid inlet port 50 is defined by a flow control valve 52 having a sanitary fitting, and is connected by and through an inlet port sleeve 54. Liquid outlet port 51 is defined by a flow control valve 56 having a sanitary fitting, and is connected by and through an outlet port sleeve 58. Liquid inlet port 50 and liquid outlet port 51 are in fluid communication with annular flow channel 40, and allow the liquid to be sterilized to be introduced and removed from apparatus 10.

A drain port 60 is attached by outlet port sleeve 58 which is held in place by a nut 62 at the downwardly directed end of apparatus 10. Drain port 60 facilitates flushing and cleaning of the annular flow channel 40 to prevent contamination. Support framework 24 is designed to enhance draining of annular flow channel 40 by angling the apparatus 10 so that fluid inlet port 50 is at a higher level than fluid outlet port 51.

In operation, vent fan 32 is powered to initiate the flow of ambient air through housing 12 to cool UV lights 26. The temperature of lights 26 is ideally kept at or near room temperature for maximum efficiency. Fan 32 remains powered throughout the sterilization process. Lights 26 are then turned on and the heat exchange cooling fluid is pumped into and through heat exchanger 44 by a conventional pumping mechanism, not shown, prior to the introduction of the liquid to be sterilized.

The liquid to be sterilized is then continuously pumped by a conventional system, not shown, into annular flow channel 40 through liquid inlet valve 52. In an apparatus with the preferred annular flow channel 40 of two mm in diameter, the preferred flow rate is approximately eight liters per minute. As the liquid flows through annular flow channel 40, the liquid is exposed to the UV radiation produced by lights 26. Further, the UV radiation that passes through the liquid and reaches heat exchange tube 44 will be reflected back through the liquid by the mirror finish of the heat exchange tube 44. The UV radiation that reaches the interior surface of housing 12 is also reflected by the mirror finish on the exterior most surface of tube 44 back toward and through the liquid. At this flow rate and channel diameter, it is anticipated that the liquid will be exposed to as dose of 200mw/sec/cm² UV radiation.

If at any time control panel 30 signals a problem with one or more lights 26, the pumping of the liquid is stopped, and lights 26 turned off. Housing 12 can then be opened by lifting section 14 and light(s) 26 accessed for repair or replacement. Once housing 12 is closed, the sterilization process may be continued.

The sterilized liquid is removed from the apparatus at liquid outlet port 51 through flow control valve 56. After the desired volume of liquid has been sterilized, the flow of liquid to annular flow channel 40 is discontinued, lights 26 turned off and the heat exchange cooling fluid and the cooling medium turned off. Apparatus 10 is then sterilized by opening drain outlet 60 and flushing annular flow channel 40 with a suitable sterilizing fluid.

From the foregoing description those skilled in the art will appreciate that all of the objects of the present invention are realized. A sterilization apparatus and method that allow continuous flow sterilization of heat sensitive liquids without increasing the temperature of the liquid to the point of damaging the liquid are disclosed. The apparatus and method are economical to construct and operate, and are simple to operate, clean and maintain.

Having described the present invention in detail, those skilled in the art will appreciate that modifications may be made of the present invention without departing from its spirit and scope. The apparatus of the present invention is particularly well suited for the sterilization of biological liquids, but is equally applicable to any application in which sterilization of a fluid is required. The construction of the housing, while presently preferred, can be modified to meet specific needs of a particular situation. The ring arrangement of elongated UV lights is particularly well suited for this application, but any arrangement of lights or alternate UV source that provides sufficient UV radiation and can be adapted to this application may be used.

In addition, while fused silica quartz is the preferred material for the outer flow ring, the outer flow tube may be constructed of any material that has sufficient strength while allowing adequate penetration of UV radiation. Likewise, while the heat exchange tube disclosed herein is an economical, simple system that effectively protects the heat sensitive liquid from raises in temperature, other systems may be substituted. Other means of cooling the tube, including but not limited to air cooling, the use of liquids other than water, and the use of a refrigerated coil may be substituted. The annular flow channel formed by two coaxial tubes is well suited for use in this apparatus, however, any liquid flow channel, regardless of geometry, that is adapted to allow irradiation of the liquid while the temperature of the liquid is controlled may be substituted.

Further, it is understood that the apparatus and method can be computer operated in part or whole by methods known to those skilled in the art.

Therefore, it is not intended that the scope of the present invention be limited to the specific examples and embodiments described herein. It is also contemplated that other modifications and applications, or equivalents thereof, will occur to those skilled in the art. It is accordingly intended that the claims set forth below shall cover all such changes, modifications, variations and other uses and applications that do not depart from the spirit and scope of the present invention as described herein.

In view of the foregoing, it will be seen that the several objects of the invention are achieved and other advantages are attained. Although the foregoing includes a description of the best mode contemplated for carrying out the invention, various modifications are conceivable.

What is claimed is:

1. An apparatus for sterilizing a fluid comprising:
a housing;
a fluid channel within the housing adapted to control the temperature of the fluid; and
an ultraviolet source disposed within the housing sufficiently close to the fluid channel to permit irradiation of the fluid as the fluid flows through the fluid channel.

2. The apparatus of claim 1 wherein the fluid channel is formed by inner and outer flow tubes arranged coaxially to form an annular channel between the inner and outer flow tubes, the outer flow tube being adapted to allow penetration of UV radiation, and the inner tube being a heat exchanger.

3. An apparatus for sterilizing a liquid comprising:
a housing;
inner and outer flow tubes arranged coaxially within the housing to form an annular flow channel between the inner and outer flow tubes, the outer flow tube being adapted to allow penetration of UV radiation, the inner tube being a heat exchanger;
at least one liquid inlet port and at least one liquid outlet port, the liquid inlet port and the liquid outlet port being in fluid communication with the annular flow channel; and
at least one UV radiation source to sterilize the liquid, the UV radiation source being located within the housing outwardly of the inner and outer flow tubes;
to thereby permit the liquid to be sterilized to be introduced through the inlet port into the annular flow channel, the liquid being irradiated by the UV radiation source as the heat exchanger controls the temperature of the liquid until the liquid is removed from the annular flow channel through the outlet port.

4. The apparatus of claim 3 wherein the housing further includes a cooling mechanism to cool the UV radiation source.

5. The apparatus of claim 3 wherein the UV radiation source is a plurality of UV lights.

6. The apparatus of claim 3 wherein the heat exchanger is a mirror finish stainless steel heat exchanger.

7. An apparatus for sterilizing a heat sensitive liquid comprising:
a housing including a cooling mechanism;
inner and outer flow tubes arranged coaxially to form an annular flow channel between the inner and outer flow tubes, the outer flow tube being adapted to allow penetration of UV radiation, the inner tube being a mirror finish stainless steel heat exchanger;
at least one fluid inlet port and at least one fluid outlet port, the fluid inlet port and the fluid outlet port being in fluid communication with the annular flow channel for passage therethrough of the fluid to be sterilized; and
a plurality of UV lights to produce UV radiation to sterilize the liquid, the UV lights being located within the housing and outside the inner and outer flow tubes;
to thereby permit the liquid to be sterilized to be introduced through the inlet port into the annular flow channel, the liquid being irradiated by the UV lights as the UV lights are cooled by the cooling mechanism, the heat exchanger controlling the temperature of the liquid until the liquid is removed from the annular flow channel.

8. The apparatus of claim 7 wherein the cooling mechanism includes at least one cooling medium inlet and at least one cooling medium outlet, to thereby permit cooling medium to be introduced into the housing to cool the UV lights.

9. The apparatus of claim 7 wherein the housing is supported by a support framework adapted to position the apparatus such that the liquid inlet port is at a higher level than the liquid outlet port.

10. The apparatus of claim 7 wherein the UV lights are a plurality of elongated UV lights arranged in a ring around the outer flow tube.

11. The apparatus of claim 7 wherein the outer flow tube is fused silica quartz.

12. An apparatus for sterilizing a heat sensitive liquid comprising:
a substantially cylindrical housing including a cooling mechanism having at least one cooling medium inlet and at least one cooling medium outlet;
inner and outer flow tubes arranged coaxially to form an annular flow channel between the inner and outer flow tubes, the outer flow tube being fused silica quartz, the inner tube being a mirror finish stainless steel heat exchanger, so that the UV radiation penetrates the quartz tube to irradiate the liquid in the annular flow channel, the UV radiation being reflected by the heat exchanger back through the liquid in the annular flow channel, the heat exchanger including at least one heat exchange fluid inlet port and at least one heat exchange fluid outlet port so that a heat exchange fluid can flow through the heat exchanger to control the temperature of the liquid being sterilized;
a plurality of elongated UV lights located within the housing and arranged in a ring around the outer flow tube; and
a housing support framework adapted to position the housing so that the liquid inlet port is at a higher level than the liquid outlet port;
to thereby permit the liquid to be sterilized to be introduced through the fluid inlet port into the annular flow channel, the liquid being irradiated by the UV lights, and the UV lights being cooled by the cooling medium as the heat exchanger controls the temperature of the liquid until the liquid is removed from the annular flow channel through the fluid outlet port.

13. The apparatus of claim 12 wherein the housing is constructed of aluminum and has a mirror polished interior, so that the UV radiation is reflected off the housing interior back toward the annular flow channel and the liquid to be sterilized.

14. The apparatus of claim 12 wherein the housing further includes a movable portion to facilitate access to the interior of the housing.

15. The apparatus of claim 12 wherein the cooling medium is ambient air blown into the cooling medium inlet, through the housing and out through the cooling medium outlet.

16. A method for sterilizing a liquid comprising:
determining sterilization parameters including an UV radiation level, a thickness for a liquid flow channel and a flow rate through the liquid flow channel necessary to sterilize the liquid;
introducing the liquid into the flow channel;
flowing the liquid through the flow channel at the determined flow rate;
irradiating the liquid in the flow channel with an UV radiation source to sterilize the liquid while simultaneously;
cooling the liquid in the flow channel to prevent degradation of the liquid; and
removing the sterilized liquid from the flow channel.

17. The method of claim 16 farther including cooling the UV radiation source.

18. A method for sterilizing a heat sensitive liquid comprising:
determining sterilization parameters including an UV radiation level, a thickness for a liquid flow channel and a flow rate through a liquid flow channel necessary to sterilize the liquid;
introducing the liquid into the flow channel, the flow channel being formed between an outer flow tube and an inner flow tube coaxially arranged relative to one another, the outer flow tube being transparent to UV radiation and the inner flow tube being a heat exhanger;
flowing the liquid through the flow channel at the determined flow rate;
irradiating the liquid in the flow channel with an UV radiation source to sterilize the liquid, the UV radiation penetrating the outer flow tube, while simultaneously;
cooling the liquid in the flow channel as the liquid flows over the heat exchanger to prevent heat degradation of the liquid; and
removing the sterilized liquid from the flow channel.

19. The method of claim 18 further including cooling the UV radiation source.
